(19) United States
(12) Patent Application Publication
(10) Pub. No.: US 2003/0093052 A1
(43) Pub. Date: May 15, 2003

(54) Absorbent article comprising an absorbent structure

(76) Inventors: Ted Guidotti, Goteborg (SE); Mikael Andersson, Goteborg (SE)

Correspondence Address: BURNS, DOANE, SWECKER & MATHIS, L.L.P., P.O. Box 1404, Alexandria, VA, 22313-1404, US

(21) Appl. No.: 10/290,326

(22) Filed: Nov. 8, 2002

Related U.S. Application Data
(60) Provisional application No. 60/331,158, filed on Nov. 9, 2001.

(51) Int. Cl. A61F 13/15

(52) U.S. Cl. 604/378; 604/385.01

(57) ABSTRACT

An absorbent article includes an absorbent structure enclosed between a surface layer (2) and a backing layer (3), where the absorbent structure has at least one movable absorption body (4, 4a) and where a transport element (24) is arranged on each movable absorption body (4, 4a). The transport element (24) is designed in such a manner that it has lower friction in a given direction against surrounding material than in other directions.

### CROSS-REFERENCE TO RELATED APPLICATIONS

The present application claims the benefit under 35 U.S.C. §119 of U.S. Provisional Application No. 60/331,158, filed in the United States on Nov. 9, 2001, the entire contents of which are hereby incorporated herein by reference.

### BACKGROUND OF THE INVENTION

1. Technical Field

The present invention relates to an absorbent article, such as a diaper, an incontinence pad, a sanitary towel or the like, comprising an absorbent structure.

2. Background Art

Conventional absorbent structures for use in absorbent articles, such as, for example, diapers, incontinence pads, sanitary towels or the like, usually have one or more absorption bodies or absorbent layers arranged one on another. The absorbent layers can have different constructions and compositions in order for the absorbent structure to have characteristics which are suitable for the absorbent article in which it is to be used. An absorbent structure for the absorption of urine suitably has a construction and material composition suitable for taking up, spreading and retaining urine, and, in the same way, a structure for the absorption of blood or menstrual fluid has characteristics which are adapted for taking up, spreading and retaining such liquids. As, for example, menstrual fluid and urine require different types of absorbent structure for optimum absorption, the structures often look different in terms of construction and material composition. In addition to suitably being adapted to the liquid to be absorbed, the structure should also be adapted to the materials which surround the structure because the various materials interact with one another during the absorption process itself. What are known as superabsorbent polymers (SAPs) are usually mixed into the absorbent structure so that its absorption capacity and ability to retain liquid are increased. A superabsorbent is a material in the form of fibers, particles, granules, film, foam or the like, which has the capacity to absorb liquid corresponding to several times the weight of the superabsorbent material itself. The superabsorbent material binds the liquid and forms a liquid-containing gel. Examples of this type of conventional absorbent structure are given in EP 0 401 189 A1, U.S. Pat. Nos. 4,610,678 A, 4,834,735 A, WO 93/15702 A1 and WO 94/10957 A1.

One problem with this type of conventional absorbent structure is that it can have problems actually taking up liquid. In the case of the absorption of urine in, for example, a diaper, it is often a matter of relatively rapid take-up of large quantities of urine in a number of discharges, or what may be referred to as wettings, which means that the diaper has to be optimized according to such a type of absorption process. The first wetting is often absorbed rapidly and without problems of leakage or run-off, as the absorbent structure is then empty and receptive to liquid. Subsequent wettings, on the other hand, are absorbed less rapidly than the first because the absorbent structure is then already partly filled with liquid, for which reason it may be difficult for the diaper to absorb all the urine sufficiently rapidly when a subsequent wetting takes place, which often results in leakage. The problem is especially marked when the absorption core contains large quantities of superabsorbents. In the case of rapid processes, what is known as gel-blocking of the superabsorbents can occur, which is a consequence of the fact that the superabsorbent swells during liquid take-up. The actual gel-blocking arises when the superabsorbents in the absorption core swell up in such a manner that they clog the free surfaces in the absorbent structure. When gel-blocking occurs, the spreading of liquid in the absorbent structure is therefore effectively prevented, and the liquid then runs off the structure instead of being absorbed. A further problem with superabsorbents in large quantities is that the absorbent structure can become unwieldy and cumbersome for the person wearing the absorbent article because the superabsorbents swell up after liquid take-up and form a jelly-like mass.

Another problem with conventional absorption bodies is that the total absorption capacity is not utilized to as great an extent as is desirable. When, for example, wetting by urine takes place in a diaper, the liquid ends up in most cases in a single place, which means that the absorbent structure has to absorb a very large amount of liquid within a very limited area. In order for this to be possible at all, the absorbent structure must effectively be capable of moving the liquid to unused parts of the structure. The movement of liquid in the structure takes place by capillary spreading and this spreading is seldom adequate to obtain good liquid distribution with a high degree of utilization of the absorbent structure. Inadequate liquid-spreading with large quantities of liquid in a limited part of the absorbent article can also lead to the article feeling uncomfortable to the wearer. The liquid weighs the article down in the crotch portion and strains the elastic in such a manner that the article hangs down on the wearer. A better distribution of the liquid over a greater part of the article would therefore result in a better moisture fit and a more comfortable absorbent article for the wearer.

WO 00/00145 A2 describes an absorbent article comprising an absorbent core, where the absorbent core can be moved by a movement means which is attached by a first part to the core and anchored by a second part to the article. The movement means consists of a moisture-sensitive material, for example a thread, which shrinks when it is exposed to moisture. One disadvantage of this type of structure is that it is dependent on moisture in order to be capable of functioning, which can be a problem if the thread does not come into contact with liquid when a wetting occurs.

### OBJECTS AND SUMMARY

An object of the present invention is to remedy the above-mentioned problems and to design an absorbent article with an absorbent structure which effectively utilizes the absorption capacity of the structure so as to afford a better fit and in order to minimize the occurrence of leakage.

An absorbent article according to the present invention is characterized in that it comprises an absorbent structure enclosed between a surface layer and a backing layer, where the absorbent structure consists of at least one movable absorption body, and in that a transport means is arranged on each movable absorption body, the transport means being designed in such a manner that it has lower friction in a given direction against surrounding material than in other directions.

In one embodiment of the present invention, an absorbent article has been produced, comprising an absorbent structure of the type referred to in the introduction, which article essentially eliminates the problems mentioned above.

The absorbent article consists of, for example, diapers, incontinence pads, sanitary towels or the like and comprises an absorbent structure for taking up liquids such as menstrual fluid, blood, urine, motions and the like. The absorbent structure is enclosed between a liquid-permeable surface layer which faces the wearer during use of the article and a liquid-impermeable backing layer facing away from the wearer. The surface layer and the backing layer have an extent in the plane which is slightly greater than the absorbent structure and therefore extend outside its edges. The article has a front portion intended to face forwards on the wearer and a rear portion intended to face backwards on the wearer. If the article consists of a diaper or the like, it can also be provided with a narrower crotch portion which is intended to be positioned in the crotch of the wearer between the legs of the latter. The absorbent article is suitably symmetrical in relation to a longitudinal axis running in the longitudinal direction of the article. The transverse direction is the direction extending across the article and is parallel or essentially parallel to a transverse axis which forms a right angle with the longitudinal axis.

The absorbent structure consists of at least one movable absorption body which is arranged in the article in such a manner that it can be moved from an original position to a position separate from the original position when the absorbent structure has been exposed to one or more wettings. In order to ensure that the absorption body is not moved before it has reached a certain saturation with liquid, it can be anchored to the article by a means of attachment arranged in such a manner in the article that the means of attachment will with great likelihood come into contact with the liquid which, when the wetting or wettings take place, is stored in the absorption body. The means of attachment is designed in such a manner that it is dissolved when it comes into contact with the liquid. Suitable means of attachment are substances which are acted on by the liquid or a component forming part of the liquid in such a manner that their adhesive capacity is lost or substantially weakened. In order to bring about movement, a transport means is arranged on the absorption body. The transport means consists of an arrangement which affords the absorption body the possibility of moving in at least one main direction. The means can consist of, for example, a layer which is arranged on the underside of the absorption body and is designed in such a manner that it has lower friction in a given direction against surrounding material than in other directions. One way of producing a layer of this type is to use fibers or lamellae which are inclined in relation to the extent of the absorption body in the longitudinal direction when it is observed from the side, and also structures consisting of stamped or shaped material layers. The fibers or the lamellae can be attached to the absorption body directly or to a supporting layer which is then attached to the absorption body.

According to a first embodiment of the invention, the absorbent structure consists of a movable absorption body. The movable absorption body is arranged in the article between the surface layer and the backing layer. The first absorption body is arranged movably in the article and is moved from its original position by a transport means consisting of, for example, inclined fibers.

According to a second embodiment of the invention, the absorbent structure comprises a movable absorption body and a fixed absorption body. In the original position, the absorption bodies overlap one another at least partly.

### BRIEF DESCRIPTION OF THE FIGURES

The invention will be described in greater detail below with reference to the illustrative embodiments shown in the drawings, in which

FIG. 1 shows an embodiment of a diaper according to the invention, seen from above;

FIG. 2 shows a section along the line II-II through the diaper in FIG. 1;

FIG. 3 shows an absorbent structure according to a first embodiment, seen from the side, and

FIG. 4 shows an absorbent structure according to a second embodiment, seen from the side.

### DETAILED DESCRIPTION OF PREFERRED EMBODIMENTS

The absorbent article shown in the figures consists of a diaper or an incontinence pad 1 and comprises an absorbent structure. The absorbent structure is enclosed between a liquid-permeable surface layer 2 and a liquid-impermeable backing layer 3.

The liquid-permeable surface layer 2 preferably consists of a material which has characteristics such as dryness and softness during use of the absorbent article because this layer lies against the body of the wearer. It is desirable for the layer to have a soft and textile-like surface which remains dry even after repeated wettings. The surface layer can consist of, for example, a non-woven material with a soft and smooth surface such as, for example, a spunbond consisting of polypropylene fibers, a meltblown material or a bonded carded fibrous material. In order to keep the surface next to the skin of the wearer dry, use can be made of a hydrophobic non-woven material which is perforated so that openings are formed in the material which are larger than the cavities between the fibers in the material. In this way, liquid can be conducted down through the perforations in the surface layer to the underlying absorption body. Other examples of materials for the surface layer could be perforated plastic films such as, for example, perforated polyethylene film.

The liquid-impermeable backing layer 3 can consist of a thin film of polyethylene (PE), polypropylene (PP) or another suitable material, a hydrophobed non-woven layer, or a laminate of a thin film and a non-woven material. This type of laminate is often used in order to obtain a soft and textile-like outer side on the backing layer. In order to produce a more airy and comfortable article, it is also possible to use breathable backing layers which prevent liquid from escaping from the absorbent article but which allow moisture to be ventilated out. These breathable backing layers can consist of single material layers or of laminates consisting of, for example, blown or cast polyethylene films which are laminated with, for example, a non-woven layer consisting of spunbond or spunbond-meltblown-spunbond (SMS).

The surface layer 2 and the backing layer 3 have a slightly greater extent in the plane than the absorbent structure 4 and extend outside its edges. The layers 2 and 3 are at least in part interconnected within the projecting portions, for example by gluing or welding using heat or ultrasound. They can also be connected to parts of the absorbent structure by, for example, glue.

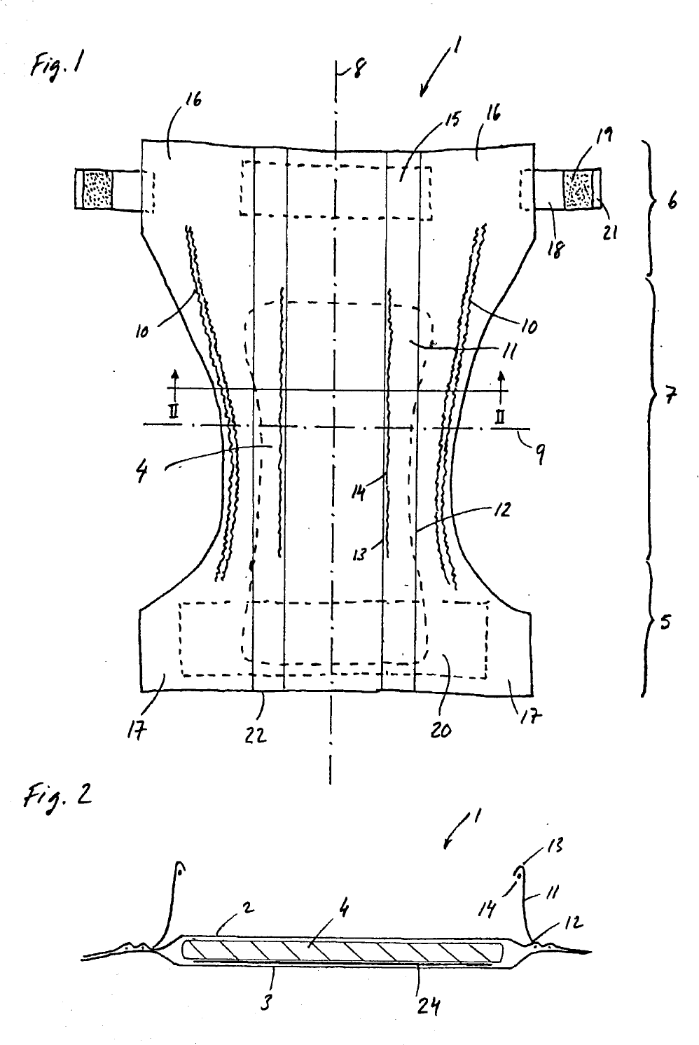

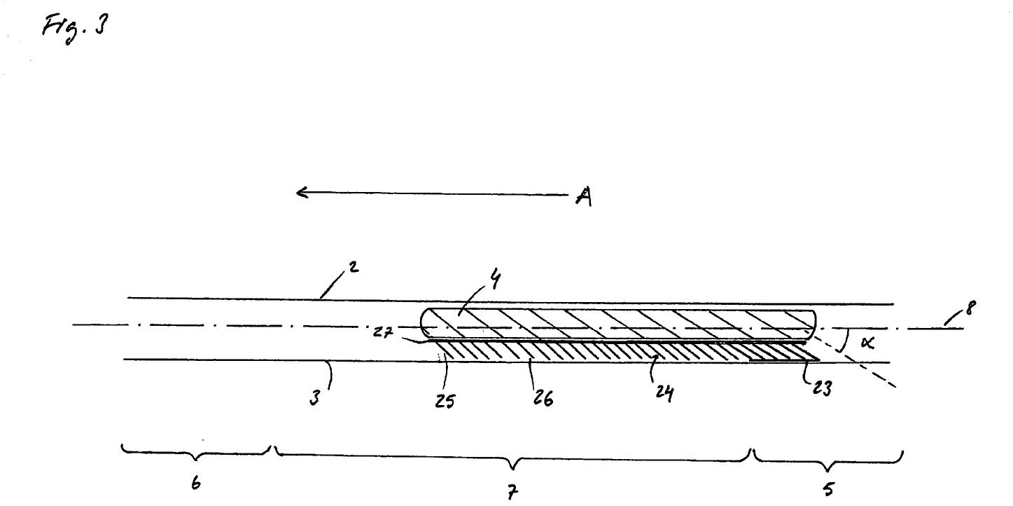

The absorbent structure consists of at least one movable absorption body 4 (see FIGS. 1-3) which can be of any conventional type. Absorption bodies are usually constructed from one or more layers of cellulose fibers, for example cellulose fluff pulp. Other materials which can be used are, for example, absorbent non-woven materials, foamed materials, synthetic fibrous materials or peat moss. In addition to cellulose fibers or other absorbent materials, the absorption bodies can also contain superabsorbent material, or superabsorbent polymers (SAPs), that is to say material in the form of fibers, particles, granules, film or the like, which has the capacity to absorb liquid corresponding to several times the weight of the superabsorbent material itself. The superabsorbent material binds the liquid and forms a liquid-containing gel. The absorption bodies can also comprise binders, shape-stabilizing components or the like. Additional absorption layers which improve the absorption characteristics can also be used, such as various types of liquid-spreading material layer or insert, what is known as wadding. The absorption bodies can be treated chemically or physically in order to modify the absorption characteristics. It is possible, for example, to provide an absorption layer with compressions in order to control the liquid flow in the absorption body. It is also possible to enclose the absorbent layer(s) in a covering made of, for example, tissue material. The absorption bodies can have a shape which is elongate in the longitudinal direction and can be, for example, essentially rectangular, T-shaped or hourglass-shaped. In a T-shaped absorption body, the transverse part is intended to face the front portion of the absorbent article during use so that it lies in the area around the abdomen of the wearer during use. An hourglass-shaped absorption body is wider in the front and rear portions than in the crotch portion in order to afford effective liquid take-up at the same time as the design makes it easier for the article to shape itself and close snugly around the wearer.

The diaper is intended to surround the lower part of the trunk of the wearer like a pair of absorbent pants. It has a front portion 5 intended to face forwards on the wearer during use, a rear portion 6 intended to face backwards on the wearer during use, and a narrower crotch portion 7 located between the front and rear portions and intended to be arranged in the crotch of the wearer between the legs of the latter.

The diaper is suitably symmetrical in relation to a longitudinal axis 8 running in the longitudinal direction of the diaper. The transverse direction of the diaper is the direction extending across the article and is parallel or essentially parallel to a transverse axis 9 which forms a right angle with the longitudinal axis 8.

Those parts on either side of the crotch portion 7 in the transverse direction which extend outside the absorbent structure are preferably provided with one or more elastic means 10 which essentially run in the longitudinal direction of the diaper. The elastic means 10 function as leg elastic and their purpose is to prevent liquid and motions leaking out through the longitudinal side edges of the diaper, thus forming outer liquid barriers. The elastic means can consist of one or more elastic threads or strips which are applied in a stretched state between the backing layer and the surface layer. Alternatively, the elastic can be arranged between the layers in an unstretched state, and the two layers instead are then stretched on application. It is also possible to arrange the elastic on the outside of the backing layer or on the inside of the surface layer.

In order further to prevent liquid or motions leaking out, the absorbent article can also be provided, on the side facing the wearer, with inner liquid barriers, or standing gathers, 11 which are attached adjacent to the longitudinal edges inside the outer liquid barriers. The inner liquid barriers 11 are preferably made from an essentially liquid-impermeable material such as, for example, a hydrophobic non-woven or a plastic film and are designed as a longitudinal web with a first edge 12 which is connected to the absorbent article and a second, free edge 13 which is intended to lie against the wearer during use of the absorbent article. The second edge is provided with one or more elastic elements 14, preferably an elastic thread, which in the contracted state gathers the free edge so that an upright barrier is formed. The inner barrier can be designed as a strip consisting of a single layer, where the free edge is turned down in order to enclose the elastic element so as to prevent direct contact between the elastic thread and the wearer. Alternatively, the barrier can be formed by two layers joined together, with the elastic thread attached at the edge of the free end between the two layers. In this case, the inner layer of the barrier can consist of an extension of the surface layer, and the outer layer can consist of an essentially liquid-impermeable material, or the inner and outer layers of the barrier can consist of one and the same material strip which is folded around the elastic thread.

The rear and/or front portions of the article can also be provided with what is known as waist elastic 15 which consists of elastic means arranged along the front and/or rear end edges so that the article surrounds the waist of the wearer in a gentle and comfortable manner. The elastic means are suitably attached between the backing layer and the surface layer by glue or by welding, for example ultrasonic welding. The elastic means can consist of one or more elastic threads which are applied in a stretched state between the layers and thus form the waist elastic. Alternatively, the elastic can be arranged between the layers in an unstretched state, and the two layers instead are then stretched on application. Another common variant of elastic which is suitable is constituted by elastic foamed materials consisting of a thin strip of, for example, polyurethane foam which can be arranged between the two layers in the same way as the elastic threads. It is also possible of course to position the elastic means for the waist elastic on the outside of the backing layer or on the inside of the surface layer.

Those parts of the rear portion of the diaper which, in the transverse direction on either side of the longitudinal axis 8, extend outside the absorption body constitute the rear side panels 16 of the diaper. In the same way, the absorbent article can be provided with front side panels 17. A fastening system for securing the absorbent article around a wearer is assigned to the front or rear side panels of the absorbent article. The fastening system consists of at least one fastening tab 18 and of a receiving part 20 for the fastening tab 18. The fastening tab 18 is intended to interconnect the rear and front portions of the absorbent article by virtue of a fastening means 19 arranged on the fastening tab 18 being fastened to the receiving part 20 which is arranged on the front or rear portion of the article. The article is preferably provided with two fastening tabs arranged on the rear side panels, one fastening tab on each rear side panel 16, and a receiving part 20 on the front portion 5 of the article, on the side facing away from the wearer during use of the diaper, that is to say on the outside of the backing layer.

The fastening tabs 18 are connected to the rear side panels 16 in the areas lying at the side edge of the side panels which runs in the longitudinal direction. The connection can be effected by, for example, glue, tape, heat sealing or welding at individual points, along lines or over continuous surface areas. The fastening tabs 18 can be attached to the backing layer of the absorbent article, the surface layer, between the backing layer and the surface layer, or be designed so that the tabs are attached so that one part of the tab lies on the outside of the backing layer and another part of the tab lies on the inside of the article, that is to say on that side of the surface layer facing the wearer.

The fastening means 19 preferably consists of a male part of a touch-and-close, or hook and loop, band. The fastening means 19 is attached to the fastening tab 18 by, for example, glue, tape, thermal connection or by another suitable means, on that part of the fastening tab 18 which faces away from the rear side panels 16 of the article and on that side of the fastening tab 18 which faces the receiving part 20 during use of the diaper. That part of the fastening tab 18 which is arranged outside the fastening means 19 in the lateral direction constitutes a gripping tab 21, the purpose of which is to facilitate application and removal of the fastening means to and from the receiving part 20.

The receiving part 20 is suitably designed as a strip which extends essentially parallel to the front end edge 22 of the diaper, that is to say in the transverse direction of the diaper, and consists of a material which is adapted for interaction with the fastening means 19. The extent of the receiving part in the transverse direction of the diaper can vary depending on model and size and can therefore extend along the entire front end edge 22 of the diaper or along a part of it. The receiving part suitably extends along a relatively great part of the front end edge 22 so that it extends over the front side panels in order that as good an adjustment as possible of the diaper to the wearer can be obtained. The receiving part 20 preferably consists of the female part (the loop part) of a hook and loop band, and the fastening means 19 of the male part (the hook part) of the hook and loop band. It would also be possible to use the hook and loop parts the other way round, but the loop part is more suitable for the receiving part because it does not have the same problems as a hook part which easily catches in clothing or other materials.

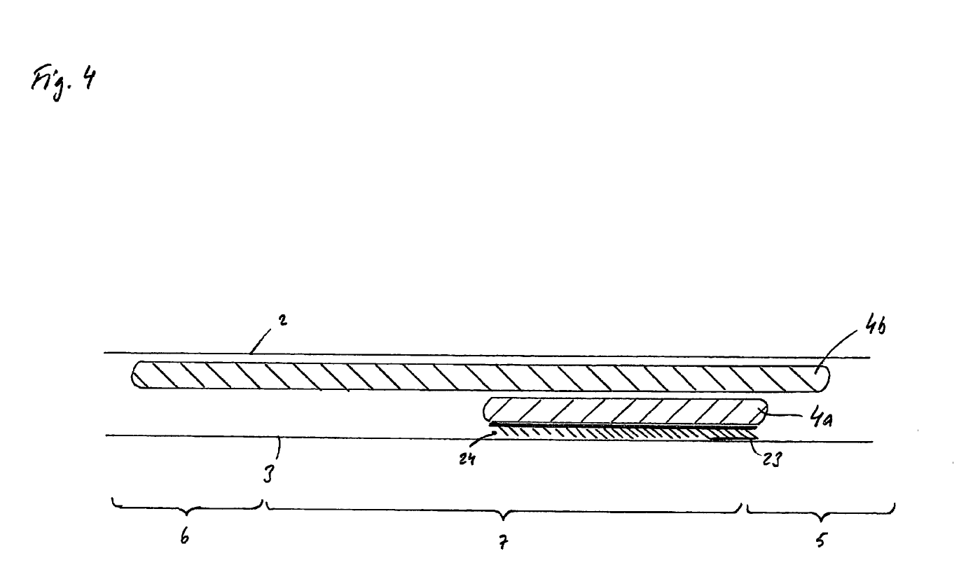

FIGS. 1-3 show a first embodiment of an absorbent structure according to the invention, FIG. 3 showing the structure seen from the side in the longitudinal direction. The absorbent structure consists of at least one movable absorption body 4 positioned in the article next to the liquid-impermeable backing layer 3, that is to say in that part of the article which faces away from the wearer during use. The fact that the absorption body 4 is movable means that it can be moved from an original position in the article to another position, for example after a certain quantity of liquid has been discharged into the absorption body. The original position of the absorption body 4 in the article 1 should suitably be such that it takes up liquid on the first wetting of the article. When the article is a diaper or an incontinence pad, the positioning should be such that the absorption body is located at least in the front part of the article so that it covers the wetting point to the greatest extent possible. Wetting point means the point or the area in the article within which the wearer discharges urine.

In order for it to be possible for the absorption body to move in the article, it is provided with a transport means 24 which acts on the absorption body so that it can move from its original position. According to the present invention, the transport means 24 consists of an arrangement which affords the absorption body 4 the possibility of moving in at least one main direction. The means can consist of, for example, a layer which is arranged on the underside of the absorption body and is designed in such a manner that it has lower friction in a given direction against surrounding material than in other directions. One way of producing a layer of this type is to use fibers or lamellae 25 which are inclined in relation to the extent of the absorption body in the longitudinal direction when it is observed from the side (see FIG. 3). The lamellae can consist of layers or strips of material such as, for example, plastic films, plastic layers, non-wovens or the like, arranged beside one another, for example so that they overlap and are inclined in relation to the extent of the absorption body in the longitudinal direction when it is observed from the side (see FIG. 3). The fibers can be made of natural or synthetic material of any suitable type.

The fibers or the lamellae form an angle a with the longitudinal axis 8 running in the longitudinal direction of the article, and the angle a suitably lies within the range 0-90°, preferably 5-85°, depending on material selection and design. The fibers or the lamellae 25 can be attached to the absorption body directly or to a supporting layer 27 which is then attached to the absorption body 4. The fibers or the lamellae are therefore anchored by one of their ends to the absorption body or to the supporting layer. The anchoring can be effected, for example, mechanically, by glue or another suitable means of attachment. In the embodiment according to FIG. 3, the fibers or the lamellae 25 extend from the absorption body through a supporting layer 27 and are inclined obliquely downwards (to the right in the figure). By virtue of the inclination, the absorption body has higher friction against the underlying surface in the direction in which the free ends 26 of the fibers or the lamellae are directed, that is to say to the right according to FIG. 3, than in the opposite direction because the fibers or the lamellae anchor or brake the absorption body in relation to the underlying material layer. When, on the other hand, the absorption body 4 is subjected to a force in a direction opposite to that in which the free ends 26 of the fibers or the lamellae are directed, the fibers or the lamellae 25 do not brake the absorption body because they 25 can be bent aside. The absorption body can then slide on the fibers or the lamellae in the direction of the force, that is to say to the left in the figure. The actual function of the transport means can be compared to that of climbing skins which are used on skis. In order for it to be possible for the absorption body to move at all, it must be subjected to a force which can act on it. An absorbent article of this type is subjected to a great many forces when it is worn by a wearer. When the wearer walks or moves, the article is acted on by forces which arise when movement of the legs, waist portion or back portion of the wearer takes place. These forces, and chiefly the forces which act on the crotch portion of the article, can be used in order to move the absorption body. The forces which act on the crotch portion of the article in the transverse direction can also act on a movable absorption body so that it is moved from its original position. If, for example, the absorption body is to be moved backwards in the longitudinal direction of the article, a layer with inclined fibers 25 can be applied to its underside so that the free fiber ends 26 point forwards. By virtue of the direction of the fibers 25, the absorption body 4 tends to be pushed backwards in the article when the movements of the wearer act on the absorbent article, in the direction A according to FIG. 3. There are many different ways of producing this type of transport means. It is possible, for example, to use flocked fibers which are inclined.

Other examples of suitable transport means 24 are, for example, fibers which are more flexible in one direction than in other directions and also fish-scale-like structures or other structures which allow controlled movement in a specific direction. This type of structure can be obtained by, for example, stamping or shaping a material layer such as a plastic film or a plastic layer. By virtue of the structure being provided with raised portions or patterns in the form of, for example, fish-scales, inclined spikes, angled strings and also ridges which have a sawtooth shape when observed from the side, it is possible to obtain lower friction in a given direction. It is suitable to apply the transport means to the entire underside of the absorption body or in smaller areas on the underside of the absorption body, for example in strips. It is also possible to apply the transport means to the backing layer of the article.

The purpose of the transport means 24 is to free unused absorption capacity so that the absorbent structure will have as high a degree of utilization as possible. By moving the absorption body 4 relative to the article, new absorption surfaces in the area around the wetting point are freed, so that the absorbent structure is ready to receive liquid on subsequent wettings. The movable absorption body is suitably reinforced, for example by a tissue layer, binder or the like, so as not to fall apart during the movement itself.

As the absorbent structure is enclosed between the surface layer 2 and the backing layer 3, it is important that the movable absorption body has a free passage in the article in order to be moved. This means that the materials which lie within the area of the movement must not impede the movement of the absorption body. Therefore, for example, the layers lying next to the absorption body in the direction of movement should not be connected to other layers or other parts of the article. On the other hand, it is suitable to connect surrounding material in the area where the absorption body has its final position after having moved from its original position because it cannot then travel further in the article.

In order to ensure that the absorption body 4 is not moved before it has been exposed to at least a first wetting, it can be anchored to the article by a means of attachment 23 which is designed in such a manner that it is dissolved when it comes into contact with liquid. Suitable means of attachment are substances which are acted on by the liquid or a component forming part of the liquid in such a manner that their adhesive capacity is lost or substantially weakened. The means of attachment can consist of substances which react to a change, a substance or a state in the environment such as, for example, moisture, enzymes, pH, temperature or salt content. Examples of such substances could be moisture-sensitive glues manufactured from starch, for example polysaccharides, or tissue structures, the bonds of which are dissolved so that the structure collapses when it comes into contact with liquid. Other possible means of attachment can consist of superabsorbent polymers (SAPs), the adhesive capacity of which disappears when the superabsorbents take up liquid, pH-sensitive terpolymers which are dissolved when a certain pH value is reached, or substances, for example CMC, which lose their binding capacity when they come into contact with a certain enzyme. The means of attachment 23 can be applied, for example, in layers, in spots or in another suitable pattern. In the embodiment according to FIG. 3, the means of attachment 23 lies on the underside of the absorption body 4 and the transport means 24 so that the absorption body is anchored to the backing layer 3. When the absorption body takes up liquid, it spreads through the absorption body and in this way gradually reaches the means of attachment 23 which, after a time, dissolves. It is possible to anchor the absorption body, using, for example, a liquid-soluble glue, to any parts or layers of the article such as, for example, the surface layer 2, the backing layer 3, and also other absorption bodies or absorbent layers. In this context, the important aspect is that the means of attachment 23 is applied in a place where the likelihood is as great as possible that it will come into contact with liquid when the absorption body has taken up liquid. Liquid-soluble glue means a glue which is dissolved by the type of liquid the absorbent article is intended to absorb, in this case urine. Examples of this type of glue are starch-based glues, as described above. When the glue is dissolved by the liquid, it loses its adhesive or anchoring capacity, which means that the absorption body 4 is no longer attached to the article but can be regarded as movable.

FIG. 4 shows a second embodiment of an absorbent structure according to the invention, seen from the side in the longitudinal direction. In this case, the absorbent structure consists of a movable absorption body 4a and a fixed absorption body 4b. The fixed absorption body 4b is positioned in the article next to the liquid-permeable surface layer 2, that is to say in that part of the article which faces the wearer during use. Under the second absorption body, the movable absorption body 4a is arranged so that it is completely or partly overlapped by the fixed absorption body 4b. As described previously, the movable absorption body is provided with a transport means 24, and the fixed absorption body 4b is suitably anchored to surrounding material. The movable absorption body 4a can be anchored to the article by a means of attachment 23.

In order further to distribute the liquid, two or more movable absorption bodies arranged one on top of another, overlapping or beside one another in the longitudinal or the transverse direction can be used instead of a single movable absorption body. When movement takes place, the various first absorption bodies can then be moved in directions which do not necessarily have to be parallel to one another, and it is therefore possible to move them to different areas of the article. In the embodiments described, the transport means 24 is arranged on the underside of the movable absorption bodies. It is also possible to arrange the transport means on the top sides or side edges of the absorption bodies. Within the scope of the invention, it is also possible to use a number of fixed absorption bodies in combination with one or more movable bodies. The movable absorption bodies can also be positioned on top of the fixed bodies in order to transport liquid away and to free new absorption capacity. A barrier layer can then be arranged between the movable and the fixed absorption body. The purpose of the barrier layer is to prevent the transport of liquid between the absorption bodies. The barrier layer can consist of, for example, an ordinary plastic film, but can also be made from a material which is dissolved by liquid in order that the absorption bodies will be ready to receive the liquid on a subsequent wetting. A barrier layer which is dissolved by liquid can be made from, for example, a starch-based film. The barrier layer can also be used for reducing the friction between the two absorption bodies so as to facilitate the movement of the movable absorption body 4a. Examples of barrier layers are given in U.S. Pat. No. 5,916,969 A, WO 92/02199 A1 and WO 99/65974 A2.

The invention is not to be regarded as being limited to the embodiments above as these are intended only to illustrate the invention. The scope of the invention also includes combining characteristics from different embodiments with one another. For example, spreading layers or what is known as wadding, consisting of, for example, non-woven materials, can be arranged on top of the absorption bodies in order to control the liquid flow in the desired direction or so as to increase the spreading of liquid before it is stored in the absorption bodies.

Although only preferred embodiments are specifically illustrated and described herein, it will be appreciated that many modifications and variations of the present invention are possible in light of the above teachings and within the purview of the appended claims without departing from the spirit and intended scope of the invention.

1. An absorbent article comprising an absorbent structure enclosed between a surface layer and a backing layer, where the absorbent structure comprises at least one movable absorption body, a transport means being arranged on each movable absorption body, wherein the transport means is designed in such a manner that it has lower friction in a given direction against surrounding material than in other directions.

2. The absorbent article according to claim 1, wherein the transport means consists of fibers or lamellae which are inclined in relation to the extent of the at least one movable absorption body in the longitudinal direction.

3. The absorbent article according to claim 2, wherein the fibers or the lamellae are attached directly to the absorption body.

4. The absorbent article according to claim 2, wherein the fibers or the lamellae are attached to a supporting layer which is attached to the absorption body.

5. The absorbent article according to claim 2, wherein the inclined fibers or lamellae form an angle (α) with the longitudinal axis running in the longitudinal direction of the article within the range 5-85°.

6. The absorbent article according to claim 1, wherein the transport means consists of a structure consisting of a stamped or shaped material layer such as a plastic film or a plastic layer.

7. The absorbent article according to claim 1, wherein the transport means is applied to the underside of the absorption body.

8. The absorbent article according to claim 1, wherein each movable absorption body is anchored to the article by a soluble means of attachment.

9. The absorbent article according to claim 8, wherein the soluble means of attachment is acted on by the liquid which is discharged into the article, or a component forming part of the liquid, in such a manner that its adhesive capacity is lost or substantially weakened.

10. The absorbent article according to claim 8, wherein the means of attachment consists of a liquid-soluble glue.

11. The absorbent article according to claim 8, wherein the means of attachment consists of a tissue structure.

12. The absorbent article according to claim 1, wherein a barrier layer is arranged between at least two absorption bodies forming part of the article.

13. The absorbent article according to claim 12, wherein the barrier layer is liquid-soluble.
